(19) United States
(12) Patent Application Publication
(10) Pub. No.: US 2019/0072564 A1
(43) Pub. Date: Mar. 7, 2019

(54) Tools for Predicting the Risk of Preterm Birth

(71) Applicants: The Regents of the University of California, Oakland, CA (US); University of Iowa Research Foundation, Iowa City, IA (US)

(72) Inventors: Laura Jelliffe, San Francisco, CA (US); Kelli Ryckman, Iowa City, IA (US); Jeffrey Murray, Iowa City, IA (US)

(21) Appl. No.: 16/075,108

(22) PCT Filed: Feb. 4, 2017
(86) PCT No.: PCT/US2017/016618

§ 371 Date: Aug. 2, 2018

Related U.S. Application Data
(60) Provisional application No. 62/291,719, filed on Feb. 5, 2016.

(51) Int. Cl. G01N 33/68

(52) U.S. Cl. CPC G01N 33/689; G01N 2800/60; G01N 2800/50; G01N 2800/368

(57) ABSTRACT

The invention is directed to methods and compositions of matter for predicting the risk of preterm birth (PTB) in a subject and administering interventions to subjects at elevated risk of PTB. The inventions provide a convenient, non-invasive, and accurate means of assessing PTB risk in a subject, and further provide a means of treating subjects in need of treatment and selecting appropriate interventions to reduce such risk. The diagnostic tools include novel panels of biomarkers and other factors which can be used to accurately predict risk of PTB across a population, wherein elevated risk is due to a variety of underlying physiological pathways and processes. In another aspect, the scope of the invention encompasses assay kits which are useful in the fast, accurate, and inexpensive prediction of PTB risk by multiplexed measurement of PTB risk factors.

### CROSS-RELATED APPLICATIONS

This application claims the benefit of priority to U.S. Provisional Application Ser. No. 62/291,719, entitled “Methods of Assessing Preterm Birth Risk,” filed Feb. 5, 2016, the contents of which are hereby incorporated by reference.

### STATEMENT REGARDING FEDERALLY SPONSORED RESEARCH OR DEVELOPMENT

This invention was made with government support under grant numbers HL101748, R01 HD057192, and R01 HD052953 awarded by the National Institutes of Health. The government has certain rights in the invention.

### BACKGROUND OF THE INVENTION

Preterm birth (PTB) is birth occurring before 37 weeks of gestation. PTB includes preterm premature rupture of membranes, preterm labor, and medical induction or cesarean section due to medical indication. PTB and its related complications are the leading cause of death in children less than five years of age and can cause life-long disability and health challenges in survivors.

Despite considerable effort, to date there are few mid-pregnancy tools for predicting preterm birth risk. The PreTRM™ test (Sera Prognostics, Salt Lake City, Utah) provides a mass spectroscopy based risk assessment. This test relies on mass spectroscopy methods rather than less expensive quantification platforms such as immunoassays. Accordingly, there remains a need in the art for inexpensive yet reliable PTB risk assessments.

Further, the underlying causes of PTB are not understood. Accordingly, there is a need in the art for tests that illuminate the physiological processes and pathways underlying PTB risk.

Identification of a well-performing and replicable prediction model would offer the opportunity for closer follow-up, monitoring, and if needed, intervention in at-risk women.

### SUMMARY OF THE INVENTION

The various embodiments of the invention are directed to methods and compositions of matter for predicting the risk of PTB in a subject. The inventions described herein provide the art with a convenient, non-invasive, and accurate means of assessing PTB risk in a subject, and further provide a means of selecting appropriate interventions to reduce such risk.

In certain embodiments, the invention provides diagnostic tools for predicting the risk of PTB. In one aspect, the diagnostic tools include novel panels of biomarkers and other factors which can be used to build predictive models for assessing the risk of PTB in a subject.

In another aspect, the methods of the invention encompass the application of novel predictive algorithms and other statistical analyses for determining the risk of PTB in a subject.

In another aspect, the methods of the invention encompass a method of treating a subject at risk of PTB. In one implementation, the selection of an appropriate treatment for a subject at risk of PTB is based on biomarker and maternal factor profiles.

In another aspect, the scope of the invention encompasses methods of assessing therapeutic treatments for alleviating the risk of PTB, or monitoring the efficacy of treatments administered to a subject.

In another aspect, the scope of the invention encompasses assay kits which are useful in the application of the methods of the invention, such as inexpensive and readily implemented immunoassay kits, as well as software, devices, and other assemblies of products that can aid in the performance of the methods described herein.

### BRIEF DESCRIPTION OF THE FIGURES

FIG. 1. depicts an ROC plot demonstrating the ability of the Model 1 PTB prediction algorithm to accurately assess PTB risk in a pool of patients.

FIG. 2. depicts biomarker and maternal factor profiles based on Panel A risk indicators, for two subjects. The profiles demonstrate how two subjects having similar PTB risks can have divergent biomarker profiles, indicating different underlying causes.

### DETAILED DESCRIPTION OF THE INVENTION

The various embodiments of the invention are directed to methods and compositions of matter for predicting the risk of PTB in a subject. The methods of the invention are, in part, based upon novel derivation of predictive relationships between certain indicators and PTB risk. Notably, the invention provides a tool for the accurate assessment of PTB risk across numerous underlying factors, providing a comprehensive and integrated means to assess PTB risk in the general population using novel combinations of indicators.

The general method of the invention is as follows:
a) a plurality of risk indicators are assessed in a subject;
b) the risk indicator assessments are input to a predictive model which predicts the risk of PTB in the subject; and
c) administering a PTB intervention to the subject if elevated PTB risk is assessed.

A “risk indicator,” as used herein is a factor that is predictive of PTB risk in a subject. Risk indicators may comprise various biomarkers, wherein the presence or abundance of the biomarker is indicative of an increased or decreased PTB risk. Risk factors may also include maternal characteristics, such as health history, health status, etc.

A “subject” as used herein will refer to a pregnant female of any species. The inventions disclosed herein are generally directed to the prediction and treatment of PTB in a human female and the description provided herein will, for convenience, reference human subjects. However, it will be understood that the scope of the invention extends to pregnant animals of other species, for example veterinary subjects and test animals.

As used herein, PTB will refer to preterm birth, also known as premature birth, being premature birth prior to the normal gestational age of delivery. For example, in human subjects, preterm birth refers to birth occurring at fewer than 37 weeks and includes preterm premature rupture of membranes, preterm labor, and medical induction or cesarean section due to medical indication

### Biomarkers

With respect to risk indicators, in some cases the risk indicator will comprise a biomarker, being a biological product present in the subject, including lipids, proteins, and nucleic acids. The selected biomarkers may be drawn from various categories, the categories being associated with different metabolic processes and pathways. In one implementation, risk indicators are drawn from the following categories: Placental Function; Lipid Status; Hormonal Status; and Immune Activity.

With respect to placental function, this may be assessed by any indicator which determines the degree or quality of placental function in the subject. One indicator of placental function is alpha fetoprotein (AFP). AFP levels may be determined in the subject by analysis of blood serum, amniotic fluid, or other samples, Elevated AFP levels above normal are associated with reduced placental function. Another indicator of placental function is Human chorionic gonadotropin (hCG). hCG helps maintain the corpus luteum during the early stages of pregnancy. Low hCG is implicated in risk of preterm birth. HCG may be measured in blood, urine, or other samples.

With respect to lipid status, various biomarkers may be used. Lipid status biomarkers include total cholesterol; low-density lipoprotein (LDL); high density lipoprotein (HDL); triglycerides; and the ratio of triglycerides to HDL.

With respect to hormonal status, various biomarkers related to hormone levels in the subject may be used. For example, progesterone status may be used as an indicator of hormone status. Low progesterone is associated with an elevated risk of PTB.

With respect to immune activity, a number of indicators may be used. The various immune biomarkers utilized in the practice of the invention include interleukins, interferons, chemokine ligands, TNF-alpha superfamily members, and growth factors.

In one implementation, immune activity is assessed by measurement of certain interleukin biomarkers, these interleukins being implicated in various immune or inflammation pathways. For example, interleukin biomarkers include: Interleukin 1 alpha (IL-1α) family members; interleukin 1 receptor 1 (IL1R1); interleukin-1 receptor antagonist (IL-1RA); glycoprotein 130 (also known as gp130, IL6ST, IL6-beta or CD130); interleukin 4 receptor (IL4R); interleukin 6 (IL-6); interleukin 7 (IL-7); interleukin 10 (IL-10), also known as human cytokine synthesis inhibitory factor (CSIF); interleukin 13 (IL-13); and interleukin 15 (IL-15); and leukemia inhibitory factor (LIF).

In one implementation, immune activity is assessed by the measurement of certain interferons. Interferon biomarkers may include biomarkers interferon A (INFA) and/or interferon B (INFB).

In one implementation, immune activity is assessed by the measurement of certain chemokine ligand biomarkers. Generally, increased expression of chemokine ligands is indicative of increased immune or inflammation pathway activity in the subject. Chemokine ligand biomarkers of the invention include: macrophage inflammatory protein-1β (MIP-1β), also known as CCL4; monocyte-chemotactic protein 3 (MCP3), also known as Chemokine ligand 7 (CCL7); epithelial-derived neutrophil-activating peptide (ENA-78), also known as chemokine ligand 5 (CXCLS); Interleukin 8 (IL8), also known as chemokine (C—X—C motif) ligand 8; monokine induced by gamma interferon (MIG), also known as chemokine (C—X—C motif) ligand 9 (CXCL9); Interferon gamma-induced protein 10 (IP-10), also known as C—X—C motif chemokine 10 (CXCL10); macrophage colony stimulating factor (MCSF); macrophage inflammatory protein 1-alpha (MIP1A), also known as chemokine (C-C motif) ligand 3 (CCL3); eotaxin family members (EOTAXIN); and regulated on activation, normal T cell expressed and secreted (RANTES), also known as Chemokine (C—C motif) ligand 5 (CCL5).

In one implementation, immune status is assessed by measurement of certain tumor necrosis factor alpha (TNFα)-related biomarkers. Generally, increased expression of members of the TNFα receptor superfamily members is indicative of increased immune or inflammation pathway activity in the subject. TNF alpha superfamily member biomarkers of the invention include: tumor necrosis factor receptor 1 (TNFR1), also known as tumor necrosis factor receptor superfamily member 1A (TNFRSF1A) and CD120a; CD40 ligand (CD40L), also known as CD154; TNF-related apoptosis-inducing ligand (TRAIL); and Fas ligand (FasL or CD95L).

In one implementation, immune status is assessed by measuring biomarkers comprising certain growth factors and their receptors. Growth factor biomarkers of the invention include: platelet-derived growth factor subunit B homodimer (PDGF-BB); nerve growth factor (NGF); vascular endothelial growth factor (VEGF); vascular endothelial growth factor receptor 1 (VEGFR1); vascular endothelial growth factor receptor 2 (VEGFR2), also known as kinase insert domain receptor (KDR); and vascular endothelial growth factor receptor 3 (VEGFR3), also known as related tyrosine kinase 4; and hepatocyte growth factor (HGF).

Additional biomarkers include: pregnancy associated plasma protein A (PAPP-A); inhibin (INH); intercellular adhesion molecule 1 (ICAM-1); and C-reactive protein (CRP).

It will be understood that the scope of the invention extends to equivalents of the biomarkers disclosed herein. A biomarker equivalent is a measurable species whose concentration is highly correlated with that of a disclosed biomarker, such that its value can serve as a proxy for the concentration of the selected biomarker. Biomarker equivalents include activators of the selected biomarker, species induced downstream of the selected biomarker, and breakdown products, conjugates, or metabolites of the selected biomarker.

### Maternal Factors

The methods of the invention may further include the use of maternal factors as PTB risk indicators. A maternal factor, as used herein, may comprise any attribute of the maternal subject. For example, maternal factors may encompass various demographic attributes of the subject, such as age, race or ethnicity, income status, etc. One maternal factor is “assistance status,” meaning the use of governmental medical assistance programs (e.g. Medicare). Maternal factors may further encompass health status factors associated with the subject. In one embodiment, body weight, or body mass index may be used as indicators of PTB risk, for example whether the subject has a body mass index of greater than 30. Likewise, the presence and/or severity of hypertension, diabetes, anemia, or other conditions may be used as PTB risk indicator factors. Maternal factors may further encompass pregnancy factors, such as the stage of pregnancy, e.g. gestational age. Another maternal factor is parity, the number of times a woman has previously carried a pregnancy to viable gestational age.

### Model Generation

In one aspect the invention provides a method of generating a predictive model to assess the risk of PTB in an individual subject based on that subject's risk indicators. The model is generated by a general process as follows: first, a panel of risk indicators is selected. Next, the risk indicator values for a first pool of women that experienced PTB during pregnancy and for a second pool of women did not experience PTB during pregnancy are then analyzed to derive mathematical relationships between risk indicator values and the probability of experiencing PTB.

The model may be derived from historical data sets comprising risk indicator values (e.g. maternal data and biomarker measurements) from a plurality of women in a population, wherein a subset of the women experienced PTB and another subset did not.

Various mathematical approaches exist for correlating multiple factors with the probability of a specified outcome. The predictive models of the invention may be generated using statistical methods such as: logistic regression analysis, linear discriminate analysis, partial least squares-discriminate analysis, multiple linear regression analysis, multivariate non-linear regression, backwards stepwise regression, threshold-based methods, tree-based methods, Pearson's correlation coefficient, Support Vector Machine, generalized additive models, supervised and unsupervised learning models, cluster analysis, and other statistical model generating methods known in the art. Subsets of the historical data may be utilized to generate, train, or validate the model, as known in the art.

The model input will comprise a risk indicator panel. The risk indicator panel is a set of risk indicators that are predictive of preterm birth risk. In one embodiment, the risk indicator panel comprises any two or more of the the risk indicators disclosed herein. In one embodiment, the panel comprises one or more risk indicator from each of the following categories: placental function, lipid status, hormonal status, and immune activity. In various embodiments, reference will be made to a “subset” of of indicators drawn from a defined panel, for example, being two, three, four, five, six, seven, eight, nine, ten, or more indicators drawn from the defined panel.

In one embodiment, the panel is Panel A, comprising AFP, hCG, LDL, Progesterone, IL-1A,IL-1RA, GP130, IL-7, IL-10, 11-15, IFNA, IFNB, MIP1B, MCP3, ENA78, IL-8, MIG, IP-10, CD40L, TNFR1, TRAIL, sFASL, PDGFBB, NGF, VEGF VEGFR2, assistance status, body mass index, hypertension status, and diabetes status. In one embodiment, the panel comprises a subset of the risk indicators of Panel A.

In one embodiment, the panel is Panel B, comprising parity, diabetes status, hypertension status, PAPP-A, AFP, TRAIL, IL-4, IL-5, IFNA, LIF, NGF, VEGF, VEGFR1, IP-10, MIP1A, RANTES, and CRP. In one embodiment, the panel comprises a subset of the risk indicators of Panel B.

In one embodiment, the panel is Panel C, comprising the risk indicators of first trimester PAPP-A, hCG, AFP, HDL, triglycerides, triglycerides: HDL, IL-6, CD40L, TRAIL, IL-13, LIF, MCSF, VEGFR1, VEGFR3, EOTAXIN, MCP-3, and MIG. In one embodiment, the a subset of the risk indicators of Panel C.

In one embodiment, the panel is Panel D, comprising the risk indicators of hypertension status, diabetes status, anemia status, assistance status, progesterone, AFP, hCG, INH, cholesterol, LDL, TNFR1, HGF, IL1R1, IL4R, VEGFR2, EOTAXIN, MIG, MIP1A, and ICAM1. In one embodiment, the a subset of the risk indicators of Panel D.

In one embodiment, the invention encompasses a method of generating a predictive model for the assessment of PTB risk in a subject using the risk indicators of a panel selected from the group consisting of Panel A, Panel B, Panel C and Panel D. In one embodiment, the invention encompasses a method of generating a predictive model for the assessment of PTB risk in a subject using panel comprising any two or more inidcators comprising a subset of the the group encompassing all indicators combined from Panel A, Panel B, Panel C, and Panel D.

The model inputs may be expressed in various forms, for example being continuous variables, for example, the concentration of a particular biomarker in the serum of the subject. The input may comprise a median fluorescence intensity value. The model inputs may comprise normalized variables. For example, a subject's biomarker levels may be expressed as a multiple of the median value of a relevant population. The model inputs may also comprise categorical, discrete, and stratified values. For example, the existence of pre-existing diabetes comprises a discreet, yes or no value. In some embodiments, discreet variables may be assigned a numeric value, e.g. no=0 and yes=1. In another example, a biomarker level may be deemed elevated or not, by comparison to a reference value (e.g. an average population value or a value observed in subjects not at elevated risk of PTB. Likewise, a biomarker value can be assigned to a stratum (e.g., low, normal, or high).

The generated model will comprise one or more equations, into which an individual subject's risk indicator values may be input to generate an output that is predictive of that subject's risk of PTB. Model output may comprise a probability score, odds score, risk categorical value (e.g. “low risk,” “moderate risk,” and “high risk,” etc.), such categories being based on statistical probabilities of PTB. The output of the predictive model may be a score, which can be compared to one or more statistical cutoff values which define PTB risk categories.

In some embodiments, the generated model will select subset of risk indicators from the input panel, eliminating those that did not have sufficient predictive value, based on selected retention cutoffs.

### PTB Risk Assessment and Intervention

The predictive tools provided herein may be used in various ways. In a first aspect, the invention encompasses a method of assessing PTB risk for a subject comprising the following steps:
obtaining the subject's risk indicator values for each risk indicator in a selected panel;
inputting the obtained risk indicator values to a predictive model based on the selected panel of risk indicators; and
calculating a PTB risk assessment for the subject using the predictive model.

In one aspect, the method further encompasses the step of administering an intervention to the subject if the subject is found to have an increased risk of PTB. In one embodiment, the selection of the intervention is guided by the indicator profile of the subject.

The first step is the acquisition of risk indicator values, i.e. obtaining medical data and biomarker measurements for each risk indicator in the panel. This step can be performed by one or more practitioners in one or more separate operations. For maternal factors, the factors can be derived by interviewing the subject, reviewing medical records, or or testing the subject, for example obtaining weight and height to calculate body mass index or measuring blood pressure to determine hypertension status. Missing values may be accounted for using statistical tools known in the art.

For biomarkers assessment, the various biomarkers of the selected panel may be quantified in a suitable biological sample derived from the subject. Samples include blood, plasma, serum, urine, saliva, interstitial fluid, biopsies, and other sample types withdrawn or otherwise derived from the subject. Conveniently, the biomarkers of the invention can be assessed in serum. Blood samples routinely drawn during prenatal care doctor visits, for example at a prenatal care doctor's visit conducted during 15-20 weeks of gestational age, may serve as a sample source.

Quantification of biomarkers in samples may be performed by any means known in the art. In various embodiments, biomarkers are quantified by immunoassay techniques. Exemplary immunoassays include enzyme-linked immunosorbant assays (ELISA). ELISA assays include, for example sandwich assays and competitive assays. Other techniques known in the art include Enzyme Multiplied Immunoassay Technique, radioimmunoassays, enzyme immunoassays, fluorescence immunoassays, western blotting, immunoprecipitation and particle-based immunoassays.

Mass spectrometry techniques may be utilized to analyze biomarker presence and/or concentration in the sample. For example, MALDI or SELDI mass spectroscopy techniques can be employed, as known in the art. Other analytical approaches include selected reaction monitoring, reverse phase liquid chromatography, size permeation (gel filtration), ion exchange, affinity, HPLC and other liquid chromatography or liquid chromatography-mass spectroscopy based techniques known in the art. Quantitative low cytometry may be used as well.

In one implementation, some, most, or all of the biomarkers of the selected panel are assessed in a single integrated assay.

The attained values for each risk indicator of the panel are then input to the predictive model. The predictive calculations of the model (as well as model generation steps described in the previous section) may be carried out by any suitable digital computer. Suitable digital computers may include portable devices, laptop and desktop computers, cloud computing systems, etc, using any standard or specialized operating system, such as a Unix, Windows™ or Linux™ based operating systems. The computer will comprise software, i.e. instructions coded on a non-transitory tangible computer-readable medium such as a memory drive or disk, which such instructions direct the calculations of model generation or predictive scoring.

Risk indicator values attained by medical personnel may be directly input to the computer or may be input remotely, for example via the internet. Biomarker measurements made on devices may be accessed by or uploaded to the computer. Medical history indicators may be retrieved from or be uploaded from medical record databases.

When all necessary values have been input, the predictive model will then calculate a predictive score indicative of the subject's PTB risk. This score may be retrieved from, transmitted from, displayed by or otherwise output by the computer. For example, the score may be printed or sent in the form of a message to a medical personnel's device, etc.

In one embodiment, the method comprises the assessment of PTB risk in the subject utilizing a predictive model that analyses a panel of indicators comprising all or or a subset of indicators from a defined panel, for example selected from the group consisting of Panel A, Panel B, Panel C , and Panel D. For example, the panel may comprise all of the markers in a single defined panel selected from the group consisting of Panel A, Panel B, Panel C ,and Panel D. Alternatively, the panel may comprise a subset of the risk indicators of a defined panel selected from the group consisting of defined panels Panel A, Panel B, Panel C, and Panel D, for example >50%, >60%, >70%, >80%, >85%, >90%, or >95% of the indicators within the selected panel. It will also be understood that hybrid panels may be utilized, wherein one or more markers from two, three, or four panels of the group consisting of Panel A, Panel B, Panel C, and Panel D are selected. It will also be understood that the panel of markers analyzed in the predictive model may also include additional markers not elucidated in a panel defined herein.

In one embodiment, the method comprises the assessment of PTB risk in the subject utilizing a predictive model that analyses an indicator panel comprising one or more markers from each of the following: placental function status, lipid status, hormone status, and immune status. In one embodiment, the panel further comprises income status, body status, hypertension status, and diabetes status. For example the panel may comprise one cervical function indicator, one or more hormone status indicators, one or more lipid status indicators, and two, three, four, five, six, or more indicators of immune status. In one embodiment, four, five, or more indicators of immune status comprises at least one indicator from each of interleukins, interferons, chemokine ligands, TNF-alpha superfamily members, and growth factors.

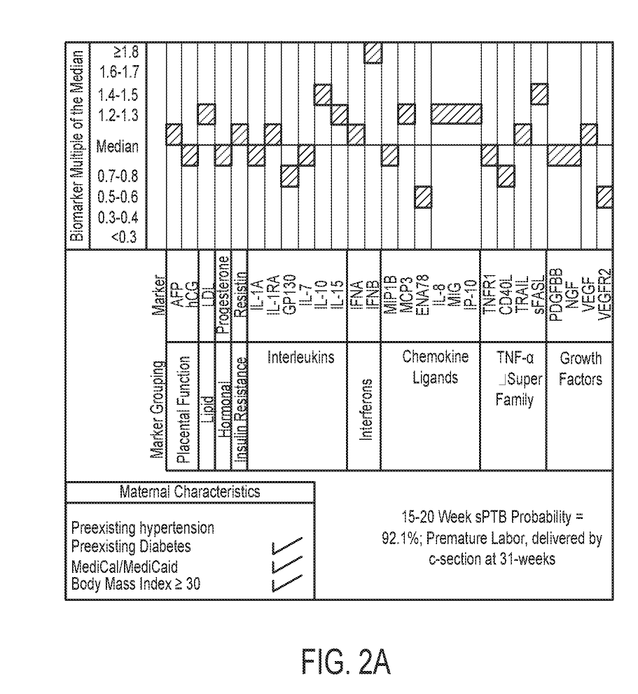

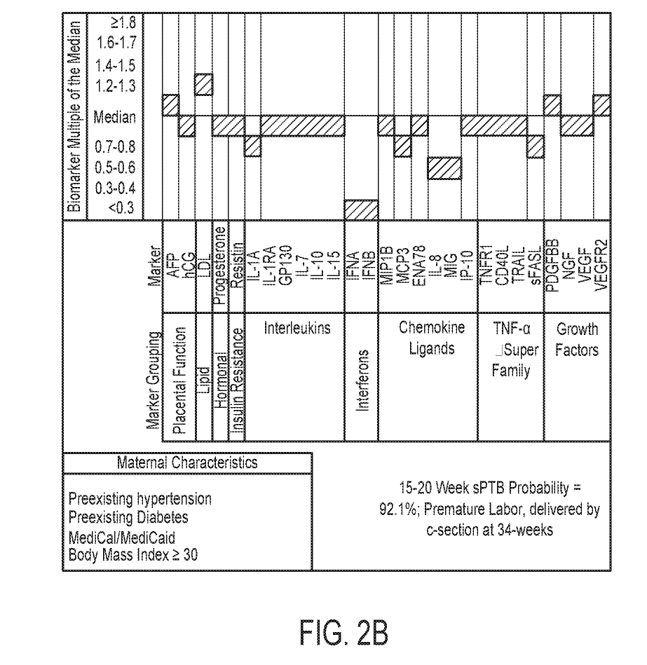

Provided herein are exemplary models which can be used to generate PTB risk predictions for subjects. Model 1 is a robust model that can predict the risk of preterm birth in pregnant subjects using the risk indicators of Panel A, generated as described in Example 1. Model 1 accurately predicts the risk of PTB in subjects experiencing both preterm premature rupture of membranes and preterm labor. For example, an ROC analysis of Model 1 attained an area under the curve score of 81% across various data sets (FIG. 2).

Model 1 coefficients for each variable of Panel A, and for significant interactions between variables, are presented in Table 1. In one embodiment, the method of the invention comprises the assessment of PTB risk in a subject using Model 1. In one embodiment, one or more of the coefficients is adjusted upwards or downwards by at least 5%, 10%, or 15%, or more.

### Table 1. Model 1 Regression Coefficients

Model 1 utilizes AFP, hCG, and LDL measurements expressed as multiple of the mean values. Discreet maternal indicators are assigned numeric value as follows: Subject using medical assistance=1, subject not using medical assistance=0; subject BMI>30=1, subject BMI<30=0; preexisting hypertension=1, no preexisting hypertension=0; and preexisting diabetes=1, no preexisting diabetes=0. All other variables are biomarker serum concentration measurements as pg/ml for placental markers, lipids, and progesterone and expressed as median fluorescence intensity (MFI) values for cytokines, chemokines and receptors.

Model 1 outputs a predictive score in the form of probability based on Equation 1 where all biomarker inputs are the log of the concentration:

PTB Probability Score=−8.1283+(1.4469*log AFP MoM)+(−0.3991*log hCG MoM)+(−0.7104*log LDL MoM)+(4.8981*log progesterone)+(−1.1834*log Il-1A)+(0.6207*log IL-1RA)+(−1.1990*log GP130)+(−1.6212*log IL-7)+(1.0055*log IL-10)+(1.9563*log IL-15)+(0.1631*log INFA)+(−0.4121*log INFB)+(−0.0767*log MIP1B)+(−1.6237*log MCP3)+(0.4761*log ENA78)+(0.2408*log IL-8)+(0.8217*log MIG)+(1.5658*log IP-10)+(0.8339*log TNFR1)+(−5.0613*log CD40L)+(9.0228*log TRAIL)+(−0.5493*log sFASL)+(1.0309*log PDGFBB)+(−17.9255*log VEGF)+(1.0385*log VEGFR2)+(3.7481*Assistance)+(0.5289*BMI)+(−12.5091*Hypertension)+(1.8859*Diabetes)+(1.8505*log TNFR1*log Progesterone)+((1.3174*log CD40L*log Progesterone)+(−5.1778*log VEGF*log Progesterone)+(−0.7364*log TRAIL*Assistance)+(−1.4070*log IL-8*Hypertension)+(5.2669*log VEGF*Hypertension), and wherein the predictive score represents the probability of PTB (e.g. a value between 0 and ‘1 or a value between 0% and 100%).

Also provided herein is Model 2. In one embodiment, the method of the invention comprises the assessment of PTB risk in a subject using Model 2, as embodied in Equation 2:

PTB probability score=−6.7601+0.9949 (log AFP MoM)−0.3583 (log hCG MoM)+0.2165 (log INH MoM)−0.5084(log TNFR1)+0.7793(log Progesterone)−0.7101 (log Cholesterol)+0.9711 (log LDL MoM)−0.2369 (log HGF)+0.3425 (log IL1R1)−0.2802(log IL4R)+0.0822 (log VEGFR2)+0.5048(log EOTAXIN)+0.1232 (log MIG)−0.2914 (log MIP1A)+0.5077 (log ICAM1)+1.3842(Hypertension value)+0.8358 (Diabetes value)+0.5719(Assistance value) +0.5426 (Anemia value) Equation 2

wherein AFP, hCG, INH values are expressed as multiples of the median values, Hypertension status=1 if the subject has any hypertension and =0 if the subject does not have any hypertension; Diabetes value=1 if the subject has any diabetes and =0 if the subject does not have any diabetes; Anemia value=1 if the subject has any anemia and =0 if the subject does not have any anemia; and Assistance value=1 if the subject has any public insurance benefit and =0 if the subject does not have any public insurance benefit. All other variables are biomarker serum concentration measurements as pg/ml for placental markers, lipids, and progesterone and median fluorescence intensity (MFI) for cytokines, chemokines and receptors.

The subject is then determined to be at elevated risk or not at elevated risk of PTB, based on selected cutoff values. The general population risk for PTB is about 10%. Accordingly, an assessed risk of 10% or greater may be deemed an elevated risk of PTB. For example, if the subject's risk score for PTB exceeds a cutoff value between 50-100%, the subject may be deemed to have an elevated risk of PTB, for example the cutoff being >55%, >60%, >70%, >75%, >85%, >90%, >95%, or higher. The determination of PTB risk may be made by the computer program, which will output or otherwise make accessible that the subject's status is elevated PTB risk. Alternatively, the determination may be made by medical personnel observing the score.

In one embodiment the method of the invention comprises the assessment process set forth above with the additional step of administering an intervention for those subjects having an elevated risk of PTB. An intervention, as used herein, means any action or treatment which is performed on or by the subject which alleviates the subject's PTB risk or which alleviates fetal harm in the event of PTB. In one embodiment, the intervention is increased monitoring of the subject, for example, monitoring of fetal health or monitoring of the cervix at periodic intervals (e.g. weekly). In one embodiment, the intervention comprises lifestyle changes, including, for example, increased rest, activity restrictions, dietary restrictions, etc. In one embodiment, the intervention is administration of cerclage (a stitch to tighten the cervix) or placement of a cervical pessary. Other interventions include, for example, administration of anti-inflammatories, administration of antibiotics, screening for infection, and administration of progesterone.

Advantageously, the risk assessment methods of the invention are able to detect PTB risk arising from a range of underlying causes. By the use of risk indicator panels that include hormone, lipid, immune, and important maternal factors, the tests provide a means of directing treatment to the underlying causes of the risk. In one aspect, the invention encompasses methods of identifying putative underlying causes in women at risk of PTB. On one embodiment, a biomarker profile is created. The biomarker profile compares the subject's biomarker measurements against population standards, such as median values, indicating the degree of variance between her biomarker measurement values and normal or median values, for example, values previously observed in women that did not experience PTB. The profile may further include maternal factor data. The profile may be presented in a graphical form, for example as a chart or drawing.

Two exemplary biomarker profiles are depicted in FIG. 2. Here, PTB assessments of two subjects (“Patient A” and “Patient B”) were performed prospectively using Model 1. Each of the subjects was found to have about a 92% probability of PTB. As it turned out, both subjects experienced PTB. However, the biomarker and maternal profiles of each patient are very different, suggesting that different underlying factors were causal for each subject's PTB.

In one embodiment, the scope of the invention encompasses methods of administering an intervention to a subject if the subject is determined to be at elevated risk of PTB, wherein the intervention is selected by analysis of the subject's biomarker panel, optionally with analysis of the subjects' and maternal indicator panel. An exemplary treatment is the administration of progesterone to subjects having lower than normal progesterone and a an elevated risk of PTB. Another exemplary treatment is the administration of anti-inflammatory compounds, for example to subjects having an elevated risk of PTB and an abnormal levels of one or more biomarkers related to immune or inflammatory pathways. In one embodiment, the intervention is monitoring for infection, if abnormal levels of one or more cytokine biomarkers is observed in the subject's profile.

The methods of the invention also provide a means to monitor the efficacy of intervention treatments. For example, a pool of subjects may be identified as having elevated risk of PTB by the methods of the invention. Women in this pool may be administered a putative treatment. Pregnancy outcomes in the treated pool can then be compared to those in a like, untreated pool to quantify the effectiveness of the putative treatment. Likewise, the methods of the invention provide a means to monitor the efficacy of a treatment. In one embodiment, the PTB risk of a subject receiving a treatment is assessed at various time points throughout pregnancy. If the subject's PTB risk decreases in response to the treatment, the treatment is deemed effective.

### Assay Kits

Provided herein are sets of risk indicators which are predictive of PTB risk. Accordingly, these discoveries enable the design of integrated assays to simultaneously measure multiple PTB risk indicators in a single sample. Provided herein are novel assay kits that can be used to facilitate the fast, inexpensive, and convenient assessment of PTB biomarker profiles in a sample. As used herein, an “assay kit” will refer to an aggregated collection of products that can be used to quantify two or more PTB biomarkers in a sample.

The assay kit will comprise a plurality of detection/quantification tools specific to each biomarker detected by the kit. Many of the biomarkers disclosed herein comprise proteins, which may be detected by immunoassays or like technologies. The detection/quantification tools may comprise capture ligands of multiple types, each directed to the selective capture of a specific biomarker in the sample. The detection/quantification tools may comprise labeling ligands of multiple types, each directed to the selective labeling of a specific biomarker in the sample, for example, comprising enzymatic, fluorescent, or chemiluminescent labels for the quantification of target species. For example, the capture and/or labeling ligands may comprise antibodies, affibodies, aptamers, or other moieties that specifically bind to a selected biomarker. The assay kit may further comprise labeled secondary antibodies, for example comprising enzymatic, fluorescent, or chemiluminescent labels labels and associated reagents.

In one embodiment, the assay kit comprises the physical elements of a quantitative e multiplex assay, for example a direct assay, an indirect assay, a sandwich assay, or a competitive assay, as known in the art, for example, an ELISA assay, wherein the assay elements enable the detection of multiple PTB risk biomarkers. Exemplary multiplex assay platforms include those described in U.S. Pat. No. 8,075,854, entitled “Microfluidic chips for rapid multiplex ELISA,” by Yang; United States Patent Publication Number US20020127740, entitled “Quantitative microfluidic biochip and method of use,” by Ho, and United States Patent Publication Number 20040241776, entitled “Multiplex enzyme-linked immunosorbent assay for detecting multiple analytes,” by Giester.

In one embodiment, the assay kit comprises a solid support to which one or more individually addressable patches of capture ligands are present, wherein the capture ligands of each patch are directed to a specific PTB biomarker. In another embodiment, individually addressable patches of absorbent or adsorbing material are present, onto which individual aliquots of sample may be immobilized. Solid supports may include, for example, a chip, wells of a microtiter plate, a bead or resin. The chip or plate of the kit may comprise a chip configured for automated reading, as known in the art.

In one embodiment, the assay kits of the invention are SELDI probes comprising capture ligands present on a solid support, which can capture the selected biomarkers from the sample and release them in response to a desorption treatment for mass spectroscopic analysis.

In one embodiment, the assay kits of the invention comprise reagents or enzymes which create quantifiable signals based on concentration dependent reactions with biomarker species in the sample. For example, lipid panel analysis may employ enzymes such as cholesterol oxidase.

Assay kits may further comprise elements such as reference standards of the biomarkers to be measured, washing solutions, buffering solutions, reagents, printed instructions for use, and containers.

In one embodiment, the assay kit of the invention is directed to the quantification of two or more PTB risk biomarkers disclosed herein. In one embodiment, the assay kit of the invention is directed to the quantification of two or more biomarkers from Panel A: AFP, hCG, LDL, Progesterone, IL-1A,IL-1RA, GP130, IL-7, IL-10, Il-15, IFNA, IFNB, MIP1B, MCP3, ENA78, IL-8, MIG, IP-10, CD40L, TNFR1, TRAIL, sFASL, PDGFBB, NGF , VEGF, and VEGFR2. In one embodiment, the assay kit of the invention is directed to the quantification of two or more biomarkers from Panel B: PAPP-A, AFP, TRAIL, IL-4, IL-5, IFNA, LIF, NGF, VEGF, VEGFR1, IP-10, MIP1A, RANTES, and CRP. In one embodiment, the assay kit of the invention is directed to the quantification of two or more biomarkers from Panel C: progesterone, PAPP-A, hCG, AFP, HDL, triglycerides, triglycerides: HDL, IL-6, CD40L, TRAIL, IL-13, LIF, MCSF, VEGFR1, VEGFR3, EOTAXIN, MCP-3, and MIG. In one embodiment, the assay kit of the invention is directed to the quantification of two or more biomarkers from Panel D: AFP, hCG, INH, cholesterol, LDL, TNFR1, HGF, IL1R1, IL4R, VEGFR2, EOTAXIN, MIG, MIP1A, and ICAM1.

### EXAMPLES

### Example 1

### Generation of Model 1 and Panel A

Model 1 was generated using multivariate backward stepwise logistic regression with consideration of two-way interactions. Four markers related to placental function were tested prospectively and 69 lipid-, hormone-, and immune-related markers in banked 15-20 gestational week serum samples collected as part of routine prenatal screening in 200 women with spontaneous PTB (100<34 weeks, 100 34-36 weeks) and 200 term controls.

So as not to lose critical information, the area under the curve (AUC) statistic was used for model selection with entry set at p<0.20 and exclusion of additional factors where AUC reduction was <0.01 after removal. Model fit was tested using a Hosmer-Lemeshow goodness of fit test. Bootstrapping with replacement was used to assess replicability (n=500 replicates).

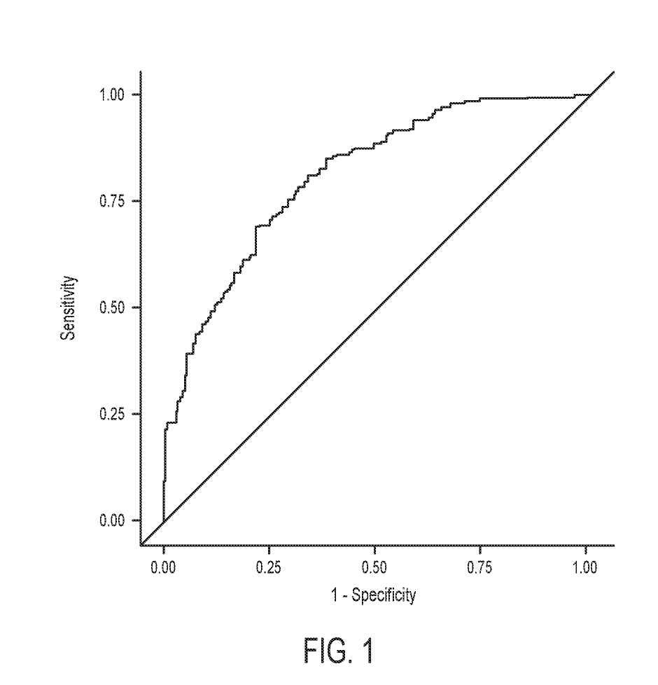

Results: The model generation step identified the risk indicators of Panel A to be predictive of PTB. The resulting Model 1 is able to identify >80% of women who went on to have a PTB (AUC=0.8110, as depicted in FIG. 1, 0.8124 in bootstrapped sample). Performance was similar in <34 and 34-36 week PTB subsets and in those with preterm premature rupture of membranes and premature labor. The Hosmer-Lemeshow test reflected good data to model fit (p=0.6190). Algorithm-driven profiles reflected individual-specific patterns across pathways of influence when similar probability scores resulted [as depicted in FIG. 2].

Conclusions: Maternal characteristics along with serum markers related to placental-, lipid-, hormone-, and immune-system function are able to predict PTB at 15 to 20 weeks with reliable accuracy.

### Example 2

### Derivation of Panel B

A subset of singleton pregnancies with prospectively measured first and second trimester serum markers available was selected. For this study 200 cases were randomly selected for closer analyses and potential specimen pulling. 173 pregnancies resulting in PTBs (74 PPROM, 99 premature labor) were selected. Controls were randomly selected at a ratio of 1:1 from the term births with frequency matching of cases and controls on body mass index (BMI) at or above 30.

Maternal indicators analyzed included race/ethnicity, maternal age, parity, preexisting diabetes, gestational diabetes, preexisting hypertension, gestational hypertension, reported smoking, participation in government health assistance for low income persons, and previous PTB.

BioMarkers examined included those tested prospectively as part of routine first and second trimester prenatal screening and markers tested on serum banked after screening. Prospectively measured first trimester analyte measurements were derived from blood samples collected between 10 weeks 0 days and 13 weeks 6 days gestation and included pregnancy associated plasma protein A (PAPP-A) and human chorionic gonadotropin (hCG). Second trimester analytes were derived from blood samples collected between 15 weeks 0 days and 20 weeks 0 days gestation in the second trimester and included alpha-fetoprotein (AFP), hCG, unconjugated estriol (uE3), and inhibin (INH). Analyte levels were measured on automated equipment. Results were entered directly into a state database along with patient information used to adjust multiple of the median (MoM) values. All analyte MoMs were adjusted for gestational age in weeks, maternal weight (as a proxy for blood volume), self-reported race/ethnicity, smoking status, and pre-existing diabetes.

Novel marker testing used residual serum used in second trimester screening (collected between 15 weeks 0 days and 20 weeks 0 days gestation). Specimens were thawed for testing. Novel markers tested included cytokines, chemokines, soluble adhesion molecules, human soluble receptors, adiponectin, lipids and c-reactive protein. To avoid error inherent in log transformation of MFI to pg/mL, analyses relied on the MFI average which was based on measurement of two aliquots tested on the same plate for each case and control. All inter-assay coefficients (CVs) were <15% across all markers and all intra-assay CVs were <10%.

Lipids (total cholesterol (TC), low-density-lipoprotein (LDL), high-density lipoprotein (HDL), and triglycerides (TGs)) and CRP were measured at using standard practices. TCs, LDLs, and HDLs were converted to MoM for gestational week at draw due to differences in levels by week at draw. No other novel marker measurement required such a conversion.

To maximize power to detect differences in between cases and controls while still allowing for some demonstration testing of resulting models, cases and controls were randomly divided into a 90% model building set (156 cases and 156 controls) and a 10% model demonstration set (17 cases and 17 controls). Logistic regression (odds ratios (ORs) and associated 95% confidence intervals (CIs)) were used to compare pregnancies resulting in early PTB (<32 weeks) to term controls in the model building set on maternal demographic and obstetric factors as well as prospectively measured and novel biomarkers. All serum measures were log transformed. Backward stepwise regression was used for final model building with criteria for staying in the model set at p<0.05 after adjustment for other factors. No restrictions on model entry were imposed, rather all factors were included in the initial model with removal of variables by highest p-value for each step. Performance was evaluated in the model building and model demonstration sets using area under the curve (AUC) statistics and their 95% confidence intervals (CIs) wherein overall performance was evaluated as well as performance by race/ethnicity grouping, maternal age, parity, preexisting diabetes, gestational diabetes, preexisting hypertension, gestational hypertension, previous PTB, and government assistance for delivery. Performance was further evaluated using receiver operator curve (ROC) derived probabilities wherein the values of predictors for a given pregnancy in the demonstration set were plotted against the ROC from the model building subset based on characteristic and serum biomarker values. Sensitivity and specificity statistics and their 95% confidence intervals were computed for ≥90, ≥80, ≥70, ≥60 and ≥50 probabilities.

The final logistic model for early PTB derived from the 90% random subset included the PTB indicators of Panel B. Three maternal indicators (parity, gestational diabetes, preexisting hypertension) and 14 biomarkers (first trimester PAPP-A, second trimester AFP, tumor necrosis factor (TNF) related apoptosis-inducing ligand (TRAIL), interleukin-4 (IL-4), IL-5, interferon alpha (IFN-α), leukemia inhibitory factor (LIF), nerve growth factor (NGF), VEGF, VEGFR1, interferon inducible protein-10 (IP-10), macrophage inflammatory protein 1-alpha (MIP1A), regulated on activation, normal t-cell expressed and secreted (RANTES), and c-reactive protein (CRP). Of particular note was the more than 10-fold increase in risk for PTB observed for women with preexisting hypertension versus not after adjusting for other characteristics and markers in the model (OR 10.7, 95% CI 2.4-48.7) and risks for PTB as high as 10-fold for log unit increases in AFP, IL-4, VEGF, IP-10 and RANTES. Substantially decreased risks per log unit increases were also observed for TRAIL, IL-5, LIF and NGF (ORs<0.1).

Considered in combination, parity, gestational diabetes, preexisting hypertension and the 14 first and second trimester markers were able to sort cases from controls with 79.4% accuracy (AUC 0.794, 95% CI 0.746-0.843). Performance was similar in women of white, Hispanic, or Asian race/ethnicity (AUCs 0.772 to 0.812), women 18-34 or ≥34 years old (AUCs 0.781 (95% CI 0.723-0.840) and 0.779 (95% CI 0.708-0.850)) and in women with gestational diabetes and not (0.777 (95% CI 0.722-0.832) and 0.879 (95% CI 0.777-0.980)). The model performed somewhat better in women who were not receiving assistance through Medi-Cal compared to those who were wherein AUCs were 0.807 (95% CI 0.741-0.873) and 0.689 (95% CI 0.604-0.774). The model derived ROC curve from the 90% subset and its resulting probabilities was highly predictive of PTB in the model building and model demonstration subsets. For example, all pregnancies determined to have PTB probabilities at or above 90 resulted in PTB in both the building and demonstration subsets. Sensitivity at this cut point was 13.5% in the model building set (95% CI 8.5-19.8) and 17.7% in the demonstration subset (95% CI 4.0-43.5). Use of a lower cut point, for example, at or above 60, resulted in better sensitivity (56.4% in the building set (95% CI 48.3-64.3) and 41.2% in the demonstration set (95% CI 18.5-67.0).

### Example 3

### Derivation of Panel C

In this, further elucidation of PTB risk indicators was performed.

Subjects: 346 singleton pregnancies without aneuploidy with expected dates of delivery in 2009 and 2010 (n=173 cases (early spontaneous PTB<32 weeks) and n=173 controls).

Biomarker Measurement: Six markers related to placental function were tested prospectively. 75 lipid and immune related markers were tested on banked second trimester (15-20 week) samples.

Model Generation: Cases and controls were divided into training and testing sets at a ratio of 3:1. Linear Discriminate Analyses (LDA) was used to identify markers in the training set that significantly contributed to sorting cases from controls. Performance of the LDA derived model was tested in both the training and testing subsets.

Results and Conclusions: The seventeen markers of Panel C were included in the final LDA model (17 out of the 81 considered). Detection in the training set was 81.74% (area under the curve (AUC) 0.8174 (95% confidence interval (CI) 0.7663-0.8686)) and 72.68% in the testing set (AUC 0.7268, 95% CI 0.6210-0.8325). Rates of model detection at set false positive rates in the training and testing set tended to be within about 10% of each other. (e.g. at a 5% FPR detection in the training set was 36.43% whereas detection in the testing set was 25.00%). The LDA model was predictive of early spontaneous PTB in women with and without hypertension or diabetes.

Findings demonstrate that in combination, placental, lipid and immune related markers may reliably identify pregnancies at increased risk for early spontaneous PTB, an so prediction models that leverage markers across multiple pathways may be robust across risk groups (e.g. those with and without hypertension or diabetes).

### Example 4

### Derivation of Model 2 and Panel D

Objective: In this study, the objective was to evaluate if second trimester serum markers related to placental function, lipids, hormone function, and the immune system can be used to assess risk for early PTB.

Study Design: Included were 400 singleton pregnancies with first and second trimester screening (100 early PTB cases (<34 completed weeks gestation) and 300 term controls (37 to 42 weeks gestation)). Four markers related to placental function were tested prospectively and 76 lipid-, inflammation/immune-, and hormone-related markers were tested on banked 15-20 week samples. Partial least squares-discriminate analysis (PLS-DA) and associated variable importance projection plots (VIPs) assessed the contribution of individual markers to group separation. Receiver operating curves (ROC) and area under the curve (AUC) statistics were used to evaluate the PLS-DA derived serum-only model and combined serum and characteristic model performance. ROC derived probabilities were used to assign level of risk.

Results: The fifteen serum markers of Panel D were included in the final PLS-DA derived predictive model including progesterone, three markers related to placental function (AFP multiple of the median (MoM), hCG MoM, INH MoM), two markers related to lipid function (cholesterol MoM and LDL MoM), and nine markers related to inflammation and immune function (TNFR1, HGF, IL1R1, IL4R, VEGFR2, EOTAXIN, MIG, MIP1A, ICAM1). The resulting model is Model 2. When combined with information about maternal hypertension, diabetes, anemia, and assistance status, serum markers Model 2 was able to sort cases and controls with 75.9% accuracy (95% confidence interval (CI) 0.701-0.817)). More than sixty-percent of women with an early PTB were identified as having at least a 3 in 10 chance of early PTB based on ROC-derived probabilities (sensitivity=61.0 (95% CI 50.7-70.6), sensitivity=81.0 (95% CI 76.5-85.6)) based on model projections.

Conclusions: Maternal characteristics and mid-pregnancy serum markers related to placental, lipid, hormone, and immune system function can be used to accurately assess risk for early PTB.

All patents, patent applications, and publications cited in this specification are herein incorporated by reference to the same extent as if each independent patent application, or publication was specifically and individually indicated to be incorporated by reference. The disclosed embodiments are presented for purposes of illustration and not limitation. While the invention has been described with reference to the described embodiments thereof, it will be appreciated by those of skill in the art that modifications can be made to the structure and elements of the invention without departing from the spirit and scope of the invention as a whole.

What is claimed is:

1-25. (canceled)

26. A method of treating preterm birth risk in a subject comprising the following steps:
obtaining risk indicator values for two or more risk indicators selected from a panel of risk indicators comprising the group consisting of: AFP, anemia status, assistance status, body mass index, CD40L, cholesterol, CRP, diabetes status, ENA78, EOTAXIN, GP130, hCG, HDL, HGF, hypertension status, ICAM1, IFNA, IFNB, IL-10, IL-13, Il-15, IL-1A, IL-1RA, IL-4, IL-5, IL-6, IL-7, IL-8, IL1R1, IL4R, INH, IP-10, LDL, LIF, MCP3, MCSF, MIG, MIP1A, MIP1B, NGF , PAPP-A, parity, PDGFBB, progesterone, RANTES, ratio of triglycerides to HDL, sFASL, TNFR1, TRAIL, triglycerides, VEGF, VEGFR1, VEGFR2, and VEGFR3;
inputting the obtained risk indicator values to a predictive model which is based on the selected panel of risk indicators;
calculating a preterm birth risk assessment for the subject using the predictive model; and
administering a preterm birth risk treatment to the subject if the subject is determined to have an elevated risk of preterm birth.

27. The method of claim 26, wherein
the panel of risk indicators comprises AFP, assistance status, body mass index, CD40L, diabetes status, ENA78, GP130, hCG, hypertension status, IFNA, IFNB, IL-10, Il-15, IL-1A, IL-1RA, IL-7, IL-8, IP-10, LDL, MCP3, MIG, MIP1B, NGF , PDGFBB, Progesterone, sFASL, TNFR1, TRAIL, VEGF, and VEGFR2.

28. The method of claim 27, wherein
the predictive model comprises the equation:
PTB Probability Score=−8.1283+(1.4469*log AFP MoM)+(−0.3991*log hCG MoM)+(−0.7104*log LDL MoM)+(4.8981*log progesterone)+(−1.1834*log Il-1A)+(0.6207*log IL-1RA)+(−1.1990*log GP130)+(−1.6212*log IL-7)+(1.0055*log IL-10)+(1.9563*log IL-15)+(0.1631*log INFA)+(−0.4121*log INFB)+(−0.0767*log MIP1B)+(−1.6237*log MCP3)+(0.4761*log ENA78)+(0.2408*log IL-8)+(0.8217*log MIG)+(1.5658*log IP-10)+(0.8339*log TNFR1)+(−5.0613*log CD40L)+(9.0228*log TRAIL)+(−0.5493*log sFASL)+(1.0309*log PDGFBB)+(−17.9255*log VEGF)+(1.0385*log VEGFR2)+(3.7481*Assistance)+(0.5289*BMI)+(−12.5091*Hypertension)+(1.8859*Diabetes)+(1.8505*log TNFR1*log Progesterone)+((1.3174*log CD40L*log Progesterone)+(−5.1778*log VEGF*log Progesterone)+(−0.7364*log TRAIL*Assistance)+(−1.4070*IL-8*Hypertension)+(5.2669*log VEGF*Hypertension);
wherein subject using medical assistance is assigned a risk indicator value of 1 and subject not using medical assistance is assigned a risk indicator value of 0;
wherein subject having a BMI>30 is assigned a risk indicator value of 1, and subject having a BMI<30 is assigned a risk indicator value of 0;
wherein subject having preexisting hypertension is assigned a risk indicator value of 1 and subject not having preexisting hypertension is assigned a value of 0;
wherein subject having preexisting preexisting diabetes is assigned a risk indicator value of 1 and subject not having preexisting diabetes is assigned a risk indicator value of 0;
wherein MoM equals multiple of the median;
wherein biomarker serum concentration measurements are measured in pg/ml for placental markers AFP and hCG, for lipid LDL and for and progesterone; and
wherein the remaining risk indicators are measured as median fluorescence intensity.

29. The method of claim 26, wherein
the selected panel of risk indicators comprises AFP, CRP, diabetes status, hypertension status, IFNA, IL-4, IL-5, IP-10, LIF, MIP1A, NGF, PAPP-A, parity, RANTES, TRAIL, VEGF, and VEGFR1.

30. The method of claim 26, wherein
the selected panel of risk indicators comprises AFP, anemia status, assistance status, CD40L, diabetes status, EOTAXIN, hCG, HDL, hypertension status, IL-13, IL-6, LIF, MCP-3, MCSF, MIG, PAPP-A, progesterone, ratio of triglycerides to HDL, TRAIL, triglycerides, VEGFR1, and VEGFR3.

31. The method of claim 26, wherein
the selected panel of risk indicators comprises AFP, cholesterol, EOTAXIN, hCG, HGF, ICAM1, IL1R1, IL4R, INH, LDL, MIG, MIP1A, TNFR1, and VEGFR2.

32. The method of claim 26, wherein
the selected panel of risk indicators comprises AFP, Anemia, Assistance, Cholesterol, Diabetes, EOTAXIN, hCG, HGF, Hypertension , ICAM1, IL1R1, IL4R, INH, LDL, MIG, MIP1A, Progesterone, TNFR1, and VEGFR2.

33. The method of claim 32, wherein
the predictive model comprises the equation:
PTB probability score=−6.7601+0.9949(log AFP MoM)−0.3583 (log hCG MoM)+0.2165 (log INH MoM)−0.5084(log TNFR1)+0.7793(log Progesterone)−0.7101 (log Cholesterol)+0.9711 (log LDL MoM)−0.2369 (log HGF)+0.3425 (log IL1R1)−0.2802(log IL4R)+0.0822 (log VEGFR2)+0.5048(log EOTAXIN)+0.1232 (log MIG)−0.2914 (log MIP1A)+0.5077 (log ICAM1)+1.3842(Hypertension value)+0.8358 (Diabetes value)+0.5719(Assistance value)+0.5426 (Anemia value);
wherein subject using medical assistance is assigned a risk indicator value of 1 and subject not using medical assistance is assigned a risk indicator value of 0;
wherein subject having anemia is assigned a risk indicator value of 1, and subject not having anemia is assigned a risk indicator value of 0;
wherein subject having preexisting hypertension is assigned a risk indicator value of 1 and subject not having preexisting hypertension is assigned a value of 0;
wherein subject having preexisting preexisting diabetes is assigned a risk indicator value of 1 and subject not having preexisting diabetes is assigned a risk indicator value of 0;
wherein MoM equals multiple of the median;
wherein biomarker serum concentration measurements are measured in pg/ml for placental markers AFP and hCG, for lipid LDL, and for progesterone; and
wherein the remaining risk indicators are measured as median fluorescence intensity.

34. The method of claim 26, wherein
the selected panel of risk indicators comprises assistance status, ENA78, EOTAXIN, GP130, IL-4, IL-5, MCSF, MIP1B, NGF, PDGFBB, RANTES, sFASL, and VEGFR3.

35. The method of claim 26, wherein
elevated is defined as a PTB risk in excess of 10%.

36. The method of claim 26, wherein
the intervention is selected from the group consisting of increased monitoring, lifestyle restrictions, progesterone administration, monitoring for infection, administration of antibiotics, administration of anti-inflammatory agents, placement of a cerclage, and placement of a cervical pessary.

37. A kit for assessing preterm birth risk biomarkers in a sample, comprising
detection elements for quantification of a panel of biomarkers comprising two or more biomarkers selected from the group consisting of CD40L, CRP, ENA78, EOTAXIN, GP130, HGF, ICAM1, IFNA, IFNB, IL-10, IL-13, 11-15, IL-1A, IL-1RA, IL-4, IL-5, IL-6, IL-7, IL-8, IL1R1, IL4R, INH, IP-10, LDL, LIF, MCP3, MCSF, MIG, MIP1A, MIP1B, NGF, PAPP-A, PDGFBB, RANTES, sFASL, TNFR1, TRAIL, VEGF, VEGFR1, VEGFR2, and VEGFR3.

38. The kit of claim 37, wherein
the panel of biomarkers comprises IL-1A, IL-1RA, GP130, IL-7, IL-10, Il-15, IFNA, IFNB, MIP1B, MCP3, ENA78, IL-8, MIG, IP-10, CD40L, TNFR1, TRAIL, sFASL, PDGFBB, NGF , VEGF, and VEGFR2.

39. The kit of claim 37, wherein
the panel of biomarkers comprises PAPP-A, TRAIL, IL-4, IL-5, IFNA, LIF, NGF, VEGF, VEGFR1, IP-10, MIP1A, RANTES, and CRP.

40. The kit of claim 37, wherein
the panel of biomarkers comprises PAPP-A, IL-6, CD40L, TRAIL, IL-13, LIF, MCSF, VEGFR1, VEGFR3, EOTAXIN, MCP-3, and MIG.

41. The kit of claim 37, wherein
the panel of biomarkers comprises INH, TNFR1, HGF, IL1R1, IL4R, VEGFR2, EOTAXIN, MIG, MIP1A, and ICAM1.

42. The kit of claim 37, wherein
the panel of biomarkers comprises EOTAXIN, hCG, HGF, ICAM1, IL1R1, IL4R, INH, MIG, MIP1A, TNFR1, and VEGFR2.

43. The kit of claim 37, wherein
the panel of biomarkers comprises ENA78, EOTAXIN, GP130, IL-4, IL-5, MCSF, MIP1B, NGF, PDGFBB, RANTES, sFASL, and VEGFR3.

44. The kit of claim 37 wherein
the detection elements comprise immunoassay capture ligands.

45. The kit of claim 37, wherein
the detection elements are immobilized on a solid support.
